(19) United States
(12) Patent Application Publication
(10) Pub. No.: US 2008/0194507 A1
(43) Pub. Date: Aug. 14, 2008

(54) Defibrotide An/Or Oligodeoxyribonucleotides For Treating Angiogenesis-Dependent Tumors

(76) Inventors: Massimo Iacobelli, Milano (IT); Gunther Eissner, Muenchen (DE); Laura Iris Ferro, Milano (IT)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/817,575

(22) PCT Filed: Feb. 27, 2006
(86) PCT No.: PCT/EP06/60304

§ 371 Date: Jan. 9, 2008

Related U.S. Application Data
(60) Provisional application No. 60/731,404, filed on Oct. 28, 2005.

(30) Foreign Application Priority Data
Mar. 3, 2005 (IT) MI2005A000336

(51) Int. Cl. A61K 31/7052; A61P 35/00

(52) U.S. Cl. 514/44

(57) ABSTRACT

The use of defibrotide and/or oligodeoxyribonucleotides having a molecular weight of 4000-10000 Dalton as an anti-tumour agent, alone or in combination with other active ingredients with anti-tumour action, is described. The oligotide may be produced by extraction from animal and/or vegetable tissues, in particular, from mammalian organs, or may be produced synthetically. The tumors which can be treated are preferably angiogenesis-dependent tumors, such as multiple myeloma or breast carcinoma.

The subject of the present invention is a method for treating a tumor-affected mammalian by administering to said mammalian an effective amount of defibrotide and/or oligotide; in particular it relates to the use of oligotide and/or defibrotide for the treatment of angiogenesis-dependent tumors.

### BACKGROUND OF THE INVENTION

Angiogenesis is a multi-step process leading to the formation of new blood vessels from pre-existing vasculature and it is necessary for primary tumor growth, invasiveness and development of metastases (20). It is normally suppressed in the adult, where angiogenesis occurs transiently only during reproduction, development and wound healing. Beyond a critical volume, a tumor cannot expand further in the absence of neovascularization (12). To promote this, a tumor must acquire the angiogenic phenotype which is the result of the net balance between positive (pro-angiogenic) and negative (anti-angiogenic) regulators (16). However, tumors are highly heterogenous in vascular architecture, differentiation, and functional blood supply (24). These differences in size of avascular preangiogenic tumors may be due in part to the capacity of tumor cells to survive under differing degrees of hypoxia (18).

Evidence for the angiogenesis-dependency of certain tumors, such as multiple myeloma, even non-solid leukemias and lymphomas (8) and (21), as well as breast (25), colorectal (7), gastric (26), prostate (9), cervix (19), hepatocellular (23), and non-small cell lung cancer (13) came from the observation that the measure of the degree of angiogenesis, the microvessel density, is an independent prognostic factor for survival in the mentioned clinical entities (17). In a recent clinical study, again in breast carcinoma, it became clear that angiogenesis-related genes are important for clinical outcome, for example the vascular endothelial cell growth factor VEGF, the VEGF receptor FLT1, and metalloproteinase MMP9 (6).

### DEFINITIONS

The term oligotide is herein used to identify any oligodeoxyribonucleotide having a molecular weight of 4000-10000 Dalton. Preferably it identifies any oligodeoxyribonucleotide having the following analytical parameters:

molecular weight (mw): 4000-10000 Dalton,

hyperchromicity (h): <10,

A+T/C+G: 1.100-1.455,

A+G/C+T: 0.800-1.160,

specific rotation: +30°-+46.8°, preferably +30°-+46.2°.

The oligotide may be produced by extraction from animal and/or vegetable tissues, in particular, from mammalian organs, or may be produced synthetically. Preferably, when produced by extraction, it will be obtained in accordance with the method described in (1), (2), and (3) which are incorporated herein by reference. The oligotide is known to be endowed with a significant anti-ischemic activity.

The term defibrotide identifies a polydeoxyribonucleotide that is obtained by extraction from animal and/or vegetable tissues but which may also be be produced synthetically; the polydesoxyribo-nucleotide is normally used in the form of an alkali-metal salt, generally a sodium salt, and generally has a molecular weight of about 45-50 kDa (CAS Registry Number: 83712-60-1). Preferably, defibrotide presents the physical/chemical characteristics described in (4) and (5), which are incorporated herein by reference.

### DESCRIPTION OF THE INVENTION

We have recently developed a model for an alternative pathway of tumor angiogenesis. In addition to the endothelial cell sprouting from pre-existing vessels, we suggest that blood borne endothelial cells might also give rise to the tumor vasculature. These endothelial-like cells (ELC) can transdifferentiate from tumor-associated dendritic cells under specific culture conditions (11). Briefly, monocytes are elutriated from leukapheresis products of healthy human blood donors and cultured in the presence of granulocyte-macrophage-colony stimulating factor (GM-CSF) and interleukin 4 (IL-4) to stimulate the differentiation of dendritic cells (DC). In addition, cells are treated with a cocktail specifically released by tumor cells (M-CSF, IL.6 and lactate, Gottfried et al., manucript submitted) to promote the outgrowth of tumor-associated dendritic cells (TuDC).

These TuDC-ELC acquire the phenotype of endothelial cells (FactorVIII related Ag, vWF) while they lose monocytic (CD14) and dendritic cell markers (CD1a). Importantly, they do not express CD34, nor CD133 or CD146 which proves that they are real transdifferentiation products and no contaminants of either circulating endothelial progenitors (CD34, CD133) or mature circulating endothelial cells (CD146).

In addition, they are able to form tube-like structures in Matrigel™, an in vitro assay of angiogenesis.

The Matrigel™ assay is one of the most popular and widely used in vitro angiogenesis assays (22). Matrigel™ is a semisolid synthetic mixture of extracellular matrix proteins which simulate the matrix that physiologically exist beneath the endothelial cell wall of a blood vessel. When the cells of question are seeded onto this matrix in microscopic chamber slides, they are activated to form tubular structures in 3-7 days, but only in the case that they have an endothelial phenotype. Therefore, this assay is suitable to show the potential capacity of cells to give rise to a tumor vasculature.

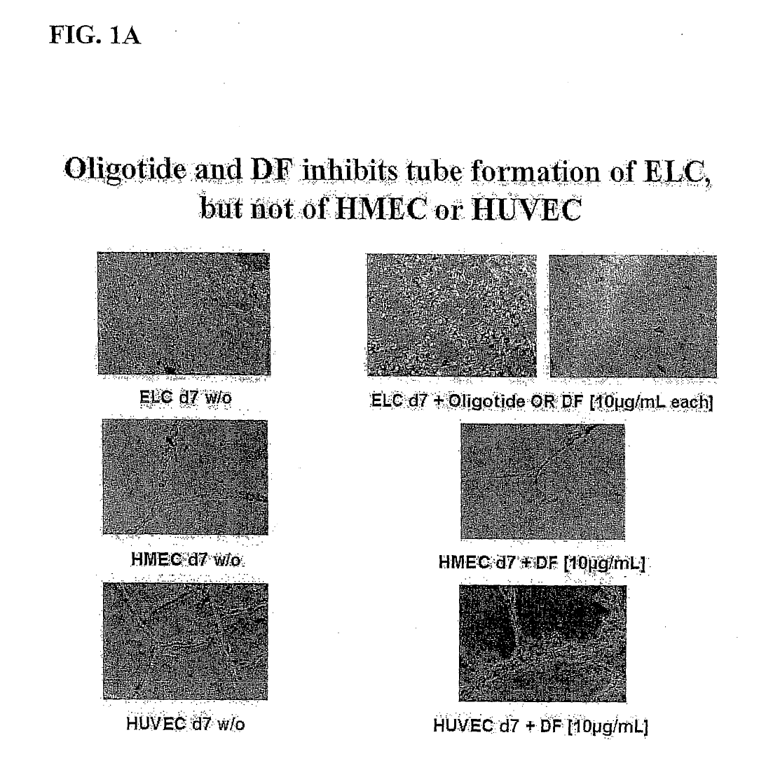

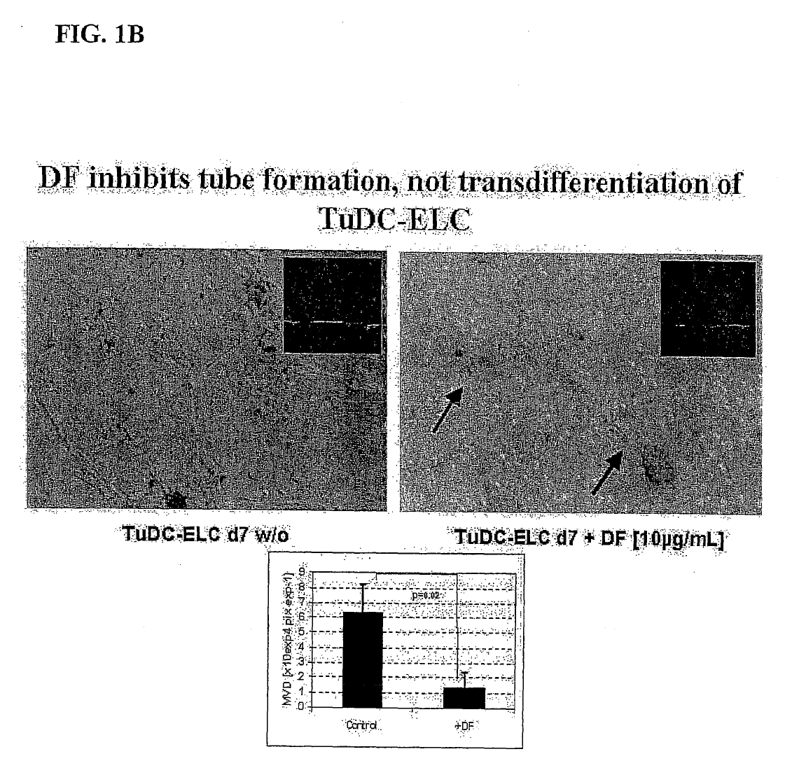

Our data data demonstrate that oligotide and/or defibrotide in clinical and subclinical concentrations can inhibit tube formation of transdifferentiating ELC (TuDC-ELC) in Matrigel™. TuDC-ELC and mature, differentiated endothelial cells, [human umbilical vene (HUVEC) or microvascular endothelial cells (HMEC) as “stable” controls] were incubated in the presence or absence of oligotide or Defibrotide (10 μg/mL each) for 7 days. Importantly, after a single addition of Defibrotide, HUVEC and HMEC are not affected in their tube formation potential, suggesting that Defibrotide and/or oligotide only target transdifferentiating endothelial cells (FIG. 1A). However, when Defibrotide was added repeatedly, it could also block angiogenesis of mature, fully differentiated endothelial cells (see below).

By the help of a complimentary software from the NIH (Image J, http://rsb.info.nih.gov/ij/), we are able to quantify these effects, the total length of tubes and the area of the photograph are assessed, the microvascular density (MVD) is then given in total length/area [pix-1]. DF significantly (p=0.02, TTEST) downregulates MVD of TuDC-ELC (FIG. 1B).

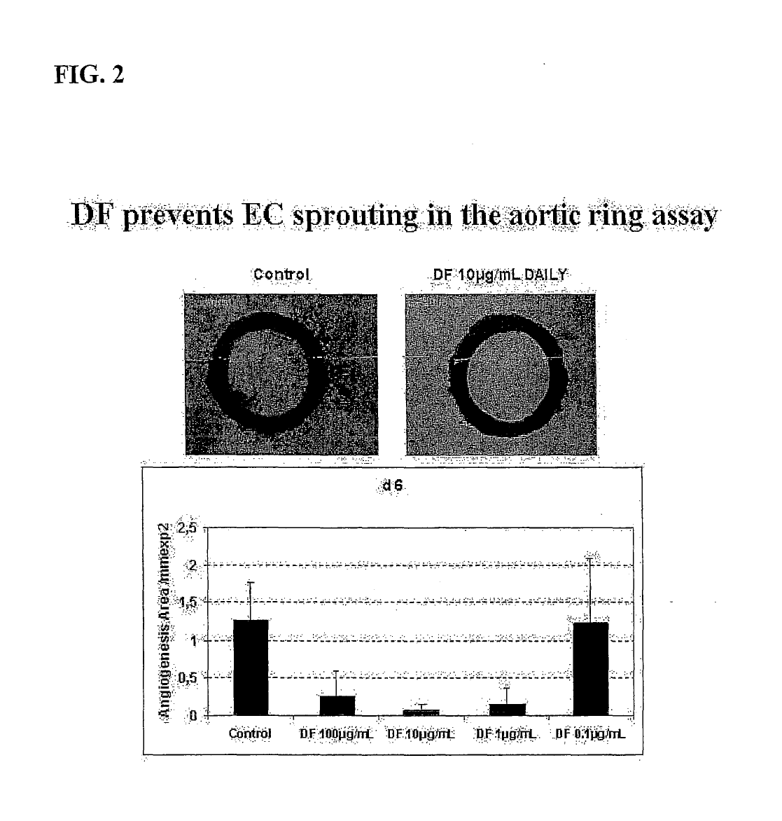

To support these data with an alternative angiogenesis assay the sprouting of rat aorta endothelial cells in Matrigel™ was prevented by nearly 100%, when DF was applied on a daily basis (FIG. 2), suggesting that DF not only acts on transdifferentiating, but also on mature, fully differentiated endothelial cells.

The aortic ring assay investigates macrovascular endothelial cells. But often, the tumor vasculature consists of microvascular endothelial cells. Therefore, a third in vitro angiogenesis assay was performed on the basis of microvascular endothelial cells vascularizing through a layer of dermal fibroblasts after 9-11 days of culture. These vessel-like structures can subsequently be visualized by staining for CD31 and vWF.

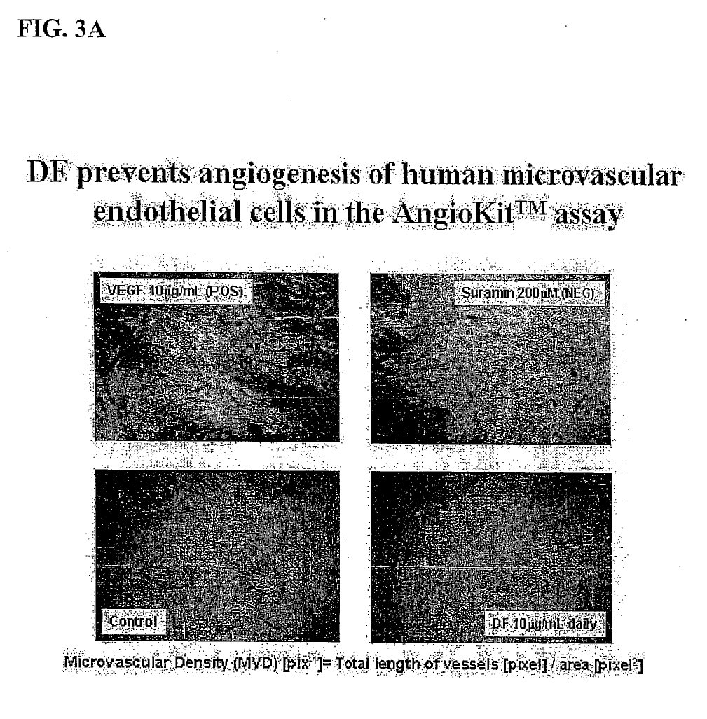

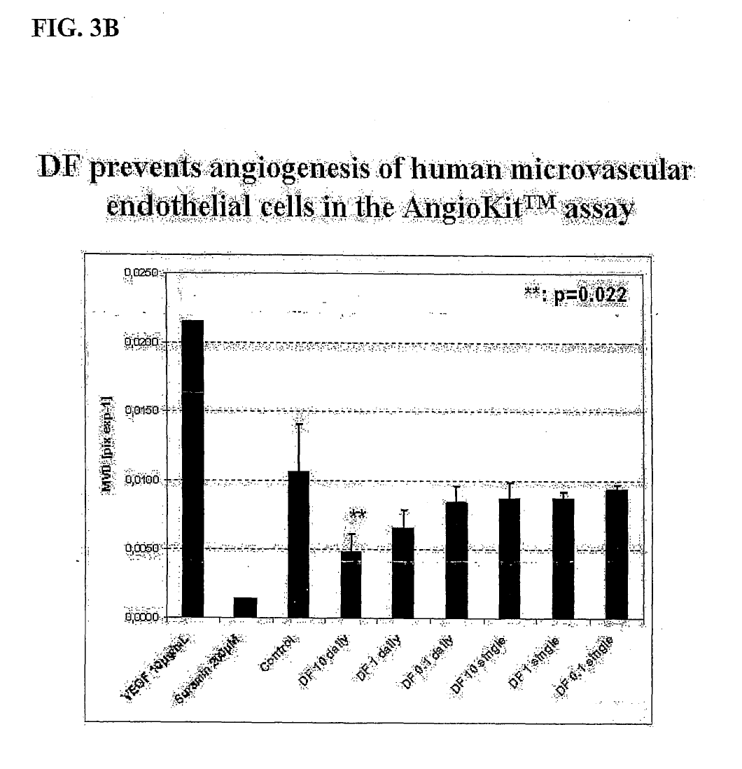

As demonstrated in FIGS. 3(A and B), DF can also block angiogenesis of human microvascular endothelial cells with a superiority for the daily application. Interestingly, concentrations around 10 μg/mL appear to be the most effective. A single application of DF could not significantly block angiogenesis.

Taken together, our data strongly suggest that defibrotide and/or oligotide can block angiogenesis of tumor-associated transdifferentiating endothelial cells and those that arise from already existing vascular cells.

It is subject to ongoing studies whether oligotide and defibrotide also inhibit angiogenesis in vivo. We are currently performing a dorsal skin chamber assay (14) that investigates the effect of defibrotide in a highly vascularized human gastric carcinoma mouse model (Xenograft system). First data clearly show that the microvascular density (MVD) of DF-treated tumors is lower than that of control tumors. This set of experiments will be reproduced in due time.

The mechanism of action by which DF can block angiogenesis remains to be elucidated, but preliminary evidence from Western Blot analyses suggest a downregulating effect of DF on activated p70S6 kinase (p-p70S6), a mitogen-activated protein kinase.

Additional evidence for the impact of p70S6 kinase was obtained from another tube formation assay with HMEC incubated in the presence or absence of the p70S6 kinase inhibitor DRB.

There are also first clinical data available for patients (pts.) having received allogeneic stem cell transplantation (SCT): In a cohort of 17 defibrotide-treated pts a striking decline in serum VEGF levels has been seen, also suggesting that defibrotide might act through growth factor withdrawal for sprouting tumor endothelial cells.

Defibrotide and oligotide are strong candidates for a therapy of angiogenesis-dependent tumors and might be used alone or in combination with other anti-angiogeneic agents, such as rapamycin (14). Interestingly, rapamycin has the negative side effect of pro-thrombotic activity (15) that could be attenuated by the simultaneous application of the anti-thrombotic and fibrionolytic defibrotide.

### REFERENCES

1. U.S. Pat. No. 5,646,127
2. U.S. Pat. No. 5,646,268
3. U.S. Pat. No. 6,046,172
4. U.S. Pat. No. 4,985,552
5. U.S. Pat. No. 5,223,609
6. 't Veer, L. J., et al. (2002) Gene expression profiling predicts clinical outcome of breast cancer. Nature, 415, 530-536.
7. Abdalla, S. A., et al. (1999) Prognostic relevance of microvessel density in colorectal tumours. Oncol. Rep., 6, 839-842.
8. Andersen, N. F., et al. (2005) Syndecan-1 and angiogenic cytokines in multiple myeloma: correlation with bone marrow angiogenesis and survival. Br. J. Haematol., 128, 210-217.
9. Bostwick, D. G. & Iczkowski, K. A. (1998) Microvessel density in prostate cancer: prognostic and therapeutic utility. Semin. Urol. Oncol., 16, 118-123.
10. Eissner, G., et al. (2002) Fludarabine induces apoptosis, activation, and allogenicity in human endothelial and epithelial cells: protective effect of defibrotide. Blood, 100, 334-340.
11. Fernandez, P. B., et al. (2001) Dendritic cells derived from peripheral monocytes express endothelial markers and in the presence of angiogenic growth factors differentiate into endothelial-like cells. Eur. J. Cell Biol., 80, 99-110.
12. Folkman, J., et al. (1971) Isolation of a tumor factor responsible for angiogenesis. J. Exp. Med., 133, 275-288.
13. Fontanini, G., et al. (1995) Microvessel count predicts metastatic disease and survival in non-small cell lung cancer. J. Pathol., 177, 57-63.
14. Guba, M., et al.(2002) Rapamycin inhibits primary and metastatic tumor growth by antiangiogenesis: involvement of vascular endothelial growth factor. Nat. Med., 8, 128-135.
15. Guba, M., et al. (2005) Rapamycin induces tumor-specific thrombosis via tissue factor in the presence of VEGF. Blood.
16. Hanahan, D. & Folkman, J. (1996) Patterns and emerging mechanisms of the angiogenic switch during tumorigenesis. Cell, 86, 353-364.
17. Hasan, J., et al. (2002) Intra-tumoural microvessel density in human solid tumours. Br. J. Cancer, 86, 1566-1577.
18. Helmlinger, G., et al. (1997) Interstitial pH and pO2 gradients in solid tumors in vivo: high-resolution measurements reveal a lack of correlation. Nat. Med., 3, 177-182.
19. Kainz, C., et al. (1995) Prognostic value of tumour microvessel density in cancer of the uterine cervix stage IB to IIB. Anticancer Res., 15, 1549-1551.
20. Morabito, A., et al. (2004) Antiangiogenic strategies, compounds, and early clinical results in breast cancer. Crit Rev. Oncol. Hematol., 49, 91-107.
21. Podar, K. & Anderson, K. C. (2005) The pathophysiologic role of VEGF in hematologic malignancies: therapeutic implications. Blood, 105, 1383-1395.
22. Staton, C. A., et al. (2004) Current methods for assaying angiogenesis in vitro and in vivo. Int. J. Exp. Pathol., 85, 233-248.
23. Sun, H. C., et al. (1999) Microvessel density of hepatocellular carcinoma: its relationship with prognosis. J. Cancer Res. Clin. Oncol., 125, 419-426.
24. Verheul, H. M., et al. (2004) Are tumours angiogenesis-dependent? J. Pathol., 202, 5-13.
25. Weidner, N., et al. (1992) Tumor angiogenesis: a new significant and independent prognostic indicator in early-stage breast carcinoma. J. Natl. Cancer Inst., 84, 1875-1887.
26. Xiangming, C., et al. (1998) Angiogenesis as an unfavorable factor related to lymph node metastasis in early gastric cancer. Ann. Surg. Oncol., 5, 585-589.

1-12. (canceled)

13. Method for the treatment of tumour which comprises the administration of defibrotide and/or oligodeoxyribonucleotides having a molecular weight of 4000-10000 Dalton alone to a patient in need of such a treatment wherein said patient is a human.

14. Method according to claim 13, wherein said oligodeoxyribonucleotides have the following analytical parameters: h<10, A+T/C+G: 1.100-1.455, A+G/C+T: 0.800-1.160, specific rotation: +30°-+46.8°.

15. Method according to claim 14, wherein the specific rotation is comprised between +30° and +46.2°.

16. Method according to claim 13, wherein said oligodeoxyribonucleotides and/or defibrotide are obtained by extraction from animal and/or vegetable tissues, preferably from mammalian organs.

17. Method according to claim 13, wherein said oligodeoxyribonucleotides and/or defibrotide are obtained synthetically.

18. Method according to claim 13, wherein said angiogenesis-dependent tumor is multiple myeloma.

19. Method according to claim 13, wherein said angiogenesis-dependent tumour is breast carcinoma.

20. Method according to claim 13, wherein said administration is intravenous.

21. Method according to claim 13, wherein said defibrotide and/or oligodeoxyribonucleotides having a molecular weight of 4000-10000 Dalton are administrated through an aqueous solution.

22. Method according to claim 13, wherein said defibrotide and/or oligodeoxyribonucleotides having a molecular weight of 4000-10000 Dalton are administrated together with customary excipients and/or adjuvants.

23. Method for the treatment of tumour which comprises the administration of defibrotide and/or oligodeoxyribonucleotides having a molecular weight of 4000-10000 Dalton in combination with rapamycin to a patient in need of such a treatment.

24. Method according to claim 23, wherein said oligodeoxyribonucleotides have the following analytical parameters: h<10, A+T/C+G: 1.100-1.455, A+G/C+T: 0.800-1.160, specific rotation: +30°-+46.8°.

25. Method according to claim 24, wherein the specific rotation is comprised between +30° and +46.20.

26. Method according to claim 23, wherein said oligodeoxyribonucleotides and/or defibrotide are obtained by extraction from animal and/or vegetable tissues, preferably from mammalian organs.

27. Method according to claim 23, wherein said oligodeoxyribonucleotides and/or defibrotide are obtained synthetically.

28. Method according to claim 23, wherein said angiogenesis-dependent tumor is multiple myeloma.

29. Method according to claim 23, wherein said angiogenesis-dependent tumour is breast carcinoma.

30. Method according to claim 23, wherein said patient is a mammalian.

31. Method according to claim 23, wherein said patient is a human.

32. Method according to claim 23, wherein said administration is intravenous.

33. Method according to claim 23, wherein said defibrotide and/or oligodeoxyribonucleotides having a molecular weight of 4000-10000 Dalton are administrated through an aqueous solution.

34. Method according to claim 23, wherein said defibrotide and/or oligodeoxyribonucleotides having a molecular weight of 4000-10000 Dalton are administrated together with customary excipients and/or adjuvants.
